(19) United States
(12) Patent Application Publication
(10) Pub. No.: US 2013/0330422 A1
(43) Pub. Date: Dec. 12, 2013

(54) METHODS FOR INCREASING INSULIN SENSITIVITY AND TREATING DIABETES WITH A BIOACTIVE CHROMIUM BINDING PEPTIDE

(71) Applicant: The Board of Trustees of the University of Alabama, Tuscaloosa, AL (US)

(72) Inventor: John B. Vincent, Tuscaloosa, AL (US)

(73) Assignee: The Board of Trustees of the University of Alabama, Tuscaloosa, AL (US)

(21) Appl. No.: 13/912,356

(22) Filed: Jun. 7, 2013

Related U.S. Application Data
(60) Provisional application No. 61/656,652, filed on Jun. 7, 2012.

(51) Int. Cl. C07K 7/06; A61K 38/28; A61K 45/06; A61K 38/08; A61K 33/24

(52) U.S. Cl. CPC C07K 7/06; A61K 38/08; A61K 33/24; A61K 45/06; A61K 38/28
USPC 424/655; 514/6.8; 514/6.9; 514/6.5

(57) ABSTRACT

Disclosed are methods and compositions related to increasing insulin sensitivity and treating diabetes.

### CROSS-REFERENCE TO RELATED APPLICATIONS

This application claims benefit of U.S. Provisional Application No. 61/656,652, filed Jun. 7, 2012, which is hereby incorporated herein by reference in its entirety.

### BACKGROUND

Diabetes is an incurable metabolic disorder characterized by high blood sugar, either because the body does not produce enough insulin, or because cells do not respond to the insulin that is produced. Currently, over 25 million people in the United States have diabetes. While about 18 million have been diagnosed, about 7 million people have been estimated to not be aware that they have the disease. According to the American Diabetes Association, diabetes is costing the US health care system an estimated $174 billion annually. More serious than the economic costs associated with diabetes are the decrease in quality of life, serious health complications, and deaths associated with diabetes. Thus, a need for new treatments for diabetes exists.

### SUMMARY

In accordance with the purposes of the disclosed materials, compounds, compositions, and methods, as embodied and broadly disclosed herein, the disclosed subject matter, in one aspect, relates to compositions and methods of preparing and using them. In a further aspect, provided herein is a method for increasing insulin sensitivity in a subject in need thereof, comprising administering a composition comprising an effective amount of a peptide to the subject, wherein the peptide comprises about 10 amino acids or less in length, wherein about 50% of the amino acids or greater are amino acids with a carboxylate side chain or amino acids with a side chain that can be converted to a carboxylate side chain, and wherein the peptide has chromium binding activity.

Also provided is a method of treating diabetes in a subject in need thereof, comprising administering a composition comprising an effective amount of a peptide to the subject, wherein the peptide comprises about 10 amino acids or less in length, wherein about 50% of the amino acids or greater are amino acids with a carboxylate side chain or amino acids with a side chain that can be converted to a carboxylate side chain, and wherein the peptide has chromium binding activity.

Further provided is a pharmaceutical composition comprising insulin in synergistic combination with a peptide comprising about 10 amino acids or less in length, wherein about 50% of the amino acids or greater are amino acids with a carboxylate side chain or amino acids with a side chain that can be converted to a carboxylate side chain, and wherein the peptide has chromium binding activity.

### BRIEF DESCRIPTION OF THE FIGURES

The accompanying Figures, which are incorporated in and constitute a part of this specification, illustrate several aspects described below.

FIG. 1 shows a negative mode MALDI/TOF PSD spectra of m/z 804 of bovine liver LMWCr peptide (A) and m/z 802, [M−H]⁻, of synthetic peptide pEEEEGDD (SEQ ID NO: 1) (B) and synthetic peptide pEEEGEDD (SEQ ID NO: 2) (C).

FIG. 2 shows a negative mode ESI/QIT (quadrapole ion trap) MS spectra of bovine liver LMWCr (A), synthetic peptide pEEEEGDD (SEQ ID NO: 1) (B), and synthetic peptide pEEEGEDD (SEQ ID NO: 2) (C).

FIG. 3 shows a CID MS/MS/MS spectra of [M−H−H₂O]⁻ from bovine liver LMWCR peptide (A), synthetic peptide pEEEEGDD (SEQ ID NO: 1) (B), and synthetic peptide pEEEGEDD (SEQ ID NO: 2) (C).

FIG. 4 shows CID of the intense peak at m/z 784, [M−H−H₂O]⁻, which dominated the MS/MS spectra for both synthetic peptides and for bovine LMWCr.

FIG. 5 shows Langmuir isotherms of Cr³⁺ binding to all the synthetic peptides.

FIG. 6 shows a Hill plot of Cr³⁺ ion binding to synthetic peptide pEEEEGDD (SEQ ID NO: 1). y corresponds to the binding number as defined by the Hill equation.

FIG. 7 is a graph showing that the endogenous chromium-binding peptide EEEEGDD (SEQ ID NO: 3) augments insulin-stimulated glucose uptake in culture myotubes.

FIG. 8A is a Western blot showing that EEEEGDD (SEQ ID NO: 3) improves insulin-stimulated phosphorylation of Akt in cultured myotubes.

FIG. 8B is a graph showing that EEEEGDD (SEQ ID NO: 3) improves insulin-stimulated phosphorylation of Akt in cultured myotubes.

FIG. 9 is a graph showing that EEEEGDD (SEQ ID NO: 3) augments insulin-stimulated glucose uptake in vivo.

### DETAILED DESCRIPTION

The materials, compounds, compositions, and methods described herein may be understood more readily by reference to the following detailed description of specific aspects of the disclosed subject matter and the Examples included therein and to the Figures.

Before the present materials, compounds, compositions, and methods are disclosed and described, it is to be understood that the aspects described below are not limited to specific peptides or methods, as such may, of course, vary. It is also to be understood that the terminology used herein is for the purpose of describing particular aspects only and is not intended to be limiting.

Also, throughout this specification, various publications are referenced. The disclosures of these publications in their entireties are hereby incorporated by reference into this application in order to more fully describe the state of the art to which the disclosed matter pertains. The references disclosed are also individually and specifically incorporated by reference herein for the material contained in them that is discussed in the sentence in which the reference is relied upon.

### DEFINITIONS

In this specification and in the claims that follow, reference will be made to a number of terms, which shall be defined to have the following meanings:

Throughout the description and claims of this specification the word “comprise” and other forms of the word, such as “comprising” and “comprises,” means including but not limited to and is not intended to exclude, for example, other additives, components, integers, or steps.

As used in the description and the appended claims, the singular forms “a,” “an,” and “the” include plural referents unless the context clearly dictates otherwise.

“Optional” or “optionally” means that the subsequently described event or circumstance can or cannot occur and that the description includes instances where the event or circumstance occurs and instances where it does not.

A “therapeutically acceptable amount” of a compound or composition of the invention, regardless of the formulation or route of administration, is that amount which elicits a desired biological response in a subject. The biological effect of the therapeutic amount may occur at and be measured at many levels in an organism. For example, the biological effect of the therapeutic amount may occur at and be measured at the cellular level by measuring the response at a receptor which binds melanocortin and/or a melanocortin analog, or the biological effect of the therapeutic amount may occur at and be measured at the system level, such as effecting an increase/decrease in the levels of insulin. The biological effect of the therapeutic amount may occur at and be measured at the organism level, such as the alleviation of a symptom(s) or progression of a disease or condition in a subject. A therapeutically acceptable amount of a compound or composition of the invention, regardless of the formulation or route of administration, may result in one or more biological responses in a subject. In the event that the compound or composition of the invention is subject to testing in an in vitro system, a therapeutically acceptable amount of the compound or composition may be viewed as that amount which gives a measurable response in the in vitro system of choice.

As used herein, the term “inhibit” refers to a decrease, whether partial or whole, in function. For example, inhibition of gene transcription or expression refers to any level of downregulation of these functions, including complete elimination of these functions. Modulation of protein activity refers to any decrease in activity, including complete elimination of activity.

As used herein, the term “diabetes” includes all known forms of diabetes, including type I and type II diabetes, as described in Abel et al., Diabetes Mellitus: A Fundamental and Clinical Text (1996) pp. 530-543.

Ranges can be expressed herein as from “about” one particular value and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed as“less than or equal to” the value, “greater than or equal to the value” and possible ranges between values are also disclosed, as appropriately understood by the skilled artisan. For example, if the value “10” is disclosed, then “less than or equal to 10” as well as “greater than or equal to 10” is also disclosed. It is also understood that throughout the application data are provided in a number of different formats and that this data represent endpoints and starting points and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Reference will now be made in detail to specific aspects of the disclosed materials, compounds, compositions, articles, and methods, examples of which are illustrated in the accompanying Examples and Figures.

### Methods

Provided herein is a method for increasing insulin sensitivity in a subject in need thereof, comprising: a) identifying a subject in need of increased insulin sensitivity; and b) administering a composition comprising an effective amount of a peptide to the subject, wherein the peptide comprises about 10 amino acids or less in length, wherein about 50% of the amino acids or greater are amino acids with a carboxylate side chain or amino acids with a side chain that can be converted to a carboxylate side chain, and wherein the peptide has chromium binding activity.

The peptide used with the methods disclosed herein can be from about four amino acids in length to about ten amino acids in length. The peptide can be, for example, four amino acids in length, 5 amino acids in length, six amino acids in length, seven amino acids in length, eight amino acids in length, nine amino acids in length, ten amino acids in length, eleven amino acids in length or twelve amino acids in length. For example, the peptide can be a peptide of about 10 amino acids or less in length comprising the formula XXXXGXX (SEQ ID NO: 4), wherein X is an amino acid with a carboxylate side chain or an amino acid with a side chain that can be converted to a carboxylate side chain. In all of the peptides disclosed herein, the amino acid can be a naturally occurring amino acid or a non-naturally occurring amino acid. For example, a naturally occurring amino acid with a carboxylate side chain can be glutamate or aspartate. A naturally occurring amino acid with a side chain that can be converted to a carboxylate side chain can be, for example, glutamine or asparagine that can be converted to glutamate and aspirate, respectively. In another example, the peptide can be a peptide comprising an amino acid sequence EEEEGDD (SEQ ID NO: 3) or a peptide consisting of amino acid sequence EEEEGDD (SEQ ID NO: 3). The peptide can also comprise or consist of the following sequences: EEEEGNN (SEQ ID NO: 5), EEEEGDN (SEQ ID NO: 6), EEEEGND (SEQ ID NO: 7), QEEEGDD (SEQ ID NO: 8), EQEEGDD (SEQ ID NO: 9), EEQEGDD (SEQ ID NO: 10), EEEQGDD (SEQ ID NO: 11), QQEEGDD (SEQ ID NO: 12), QEEQGDD (SEQ ID NO: 13), EQQEGDD (SEQ ID NO: 14), EQEQGDD (SEQ ID NO: 15), EEQQGDD (SEQ ID NO: 16), QQQEGDD (SEQ ID NO: 17), EQQQGDD (SEQ ID NO: 18), pEEEEGNN (SEQ ID NO: 19), pEEEEGDN (SEQ ID NO: 20), pEEEEGND (SEQ ID NO: 21), pQEEEGDD (SEQ ID NO: 22), pEQEEGDD (SEQ ID NO: 23), pEEQEGDD (SEQ ID NO: 24), pEEEQGDD (SEQ ID NO: 25), pQQEEGDD (SEQ ID NO: 26), pQEEQGDD (SEQ ID NO: 27), pEQQEGDD (SEQ ID NO: 28), pEQEQGDD (SEQ ID NO: 29), pEEQQGDD (SEQ ID NO: 30), pQQQEGDD (SEQ ID NO: 31), pEQQQGDD (SEQ ID NO: 32), DEEEGDE (SEQ ID NO: 33), EDEEGDE (SEQ ID NO: 34), EEDEGDE (SEQ ID NO:35), EEEDGDE (SEQ ID NO: 36), DEEEGED (SEQ ID NO: 37), EDEEGED (SEQ ID NO: 38), EEDEGED (SEQ ID NO: 39) or EEEDGED (SEQ ID NO: 40). Any of the peptides disclosed herein can be used in the methods set forth below.

In one example, the peptides disclosed herein do not comprise pyroglutamate. Pyroglutamate is formed during the removal of chromium from LMWCr. It was found that when the peptide has glutamate as the first amino acid, insulin sensitivity was increased. There are several important differences between peptides comprising pyroglutamate versus glutamate. Pyroglutamate is a cyclic amino acid found at the N termini of some proteins and biological peptides. Formation occurs through the rearrangement of the originally synthesized glutamate residue at the amino terminal position. Pyroglutamate has a different shape due to cyclization, and is neutral in charge, whereas glutamate is negatively charged and has a more extended shape. This is significant, because pyroglutamate binds chromium differently, and the difference in charge between glutamate and pyroglutamate affects the ability of the peptide to be absorbed.

Since low molecular weight chromium-binding substance has pyroglutamate at the amino terminal of the peptide in its isolated form, it was surprising that peptides comprising glutamate (such as SEQ ID NO: 3) rather than pyroglutamate at the amino terminal had biological activity at all, much less the ability to increase insulin sensitivity. Further provided is a method for increasing insulin sensitivity in a subject in need thereof, comprising: a) identifying a subject in need of increased insulin sensitivity; and b) administering a composition comprising an effective amount of a peptide disclosed herein. For example, a peptide comprising or consisting of SEQ ID NO: 3 (EEEEGDD) can be administered to the subject. Also provided is a method for increasing insulin sensitivity in a subject in need thereof, comprising administering a composition comprising an effective amount of a peptide disclosed herein to the subject, wherein the effective amount of the peptide increases glucose uptake, increases signaling and/or decreases insulin resistance in the subject. These methods can further comprise administering an effective amount of chromium to the subject. For example, a chromium(III) complex represented by the formula [Cr₃O(O₂CCH₂CH₃)₆(H₂O)³]⁺ can be administered to the subject (See U.S. Pat. No. 6,444,381, incorporated herein by this reference in its entirety). Chromium can be administered to the subject concurrently with any of the peptides disclosed herein, for example, with a peptide comprising or consisting of SEQ ID NO: 3. Chromium can also be administered before or after administration of any of the peptides disclosed herein.

These methods can optionally comprise the step of diagnosing the subject with decreased insulin sensitivity or diagnosing the subject with insulin resistance. These methods can also optionally comprise the step of diagnosing the subject with diabetes.

As utilized herein, insulin sensitivity refers to tissue responsiveness to insulin, meaning how successfully the insulin receptor operates to clear glucose from circulation. In the case of optimal insulin sensitivity, after a high sugar meal, insulin rises sharply, pushing glucose into tissues rapidly before dissipating. In the case of poor insulin sensitivity, however, insulin's elevation is sustained due to an inability to force glucose into muscle tissues. Abnormally low insulin sensitivity is called insulin resistance. In this case, tissues resist the activity of insulin on a regular basis, and the ability to remove glucose from circulation is limited. Subjects with diabetes or a pre-diabetic condition can have decreased insulin sensitivity or insulin resistance. A subject with a pre-diabetic condition has blood glucose levels that are higher than normal but not yet high enough to be diagnosed as diabetes. As utilized herein, diabetes includes, but is not limited to, all diabetic conditions, including, without limitation, diabetes mellitus, genetic diabetes, type 1 diabetes, type 2 diabetes, and gestational diabetes. Subjects with a cardiovascular condition, cancer (for example, and not to be limiting, colorectal cancer, liver cancer and pancreatic cancer), high cholesterol, high blood pressure and/or oxidative stress can also have decreased insulin sensitivity or insulin resistance. Therefore, the methods set forth herein can be used to increase insulin sensitivity in those subjects as well.

Numerous methods are known in the art for assessing insulin sensitivity (See, for example, McAuley et at “Diagnosing insulin resistance in the general population,” Diabetes Care 24:460-464 (2001)). For example, the Hyperinsulinemic-euglycemic clamp technique can be used. This clamp technique requires a steady IV infusion of insulin to be administered in one arm. The serum glucose level is clamped at a normal fasting concentration by administering a variable IV glucose infusion in the other arm. Numerous blood samplings are then taken to monitor serum glucose so that a steady fasting level can be maintained. The degree of insulin resistance should be inversely proportional to the glucose uptake by target tissues during the procedure. In other words, the less glucose that's taken up by tissues during the procedure, the more insulin resistant a patient is.

The Insulin sensitivity test (IST) can also be used. IST involves IV infusion of a defined glucose load and a fixed-rate infusion of insulin over approximately 3 hours. Somatostatin may be infused simultaneously to prevent insulin secretion, inhibit hepatic gluconeogenesis, and delay secretion of counter-regulatory hormones, particularly glucagon, growth hormone, cortisol, and catecholamines. Fewer blood samples are required for this test, compared to clamp techniques. The mean plasma glucose concentration over the last 30 minutes of the test reflects insulin sensitivity. An insulin tolerance test (ITT) can also be used. ITT measures the decline in serum glucose after an IV bolus of regular insulin (0.1-0.5 U/kg) is administered. Several insulin and glucose levels are sampled over the following 15 minutes (depending on the protocol used). The ITT primarily measures insulin-stimulated uptake of glucose into Skeletal muscle.

An increase in insulin sensitivity can be about a 10%, 20%, 30%, 40%, 50%, 60%, 70%, 80%, 90%, 100%, 200%, 300% or about a 400% increase or greater as compared to a control subject. The control subject can be the same subject prior to administration of a peptide disclosed herein. The control subject can be the same subject after administration of insulin or another anti-diabetic agent(s), but before administration of a peptide disclosed herein. The increase in insulin sensitivity can also be an increase that results in normal insulin sensitivity for the subject. Normal ranges for insulin sensitivity in a general population have been published for persons with a body mass index below 30 kg/m² and for obese subjects (BMI>30 kg/m²) at 0.026 to 0.085 mmol/L per minute and 0.012 to 0.017 mmol/L per minute, respectively. The increase in insulin sensitivity can also be an increase that results in a decrease in the amount of an anti-diabetic agent that is administered to the subject as compared to the amount of the anti-diabetic that was administered to the subject prior to administration of a peptide disclosed herein, for example, a peptide comprising or consisting of SEQ ID NO: 3.

An increase in insulin sensitivity in a pre-diabetic subject can prevent the subject from becoming diabetic. Therefore, administration of a peptide disclosed herein to a pre-diabetic subject prior to the subject needing anti-diabetic therapy, for example, insulin therapy, can reduce the likelihood that the subject will have to undergo insulin therapy. Also, if the administration of the peptide causes a sufficient increase in insulin sensitivity, patients in the early stages of diabetes could potentially forgo anti-diabetic treatment or obtain lower dosages of the anti-diabetic treatment, thus avoiding unwanted side effects.

Further provided herein is a method of treating diabetes in a subject in need thereof, comprising administering a composition comprising an effective amount of a peptide to the subject, wherein the peptide comprises about 10 amino acids or less in length, wherein about 50% of the amino acids or greater are amino acids with a carboxylate side chain or amino acids with a side chain that can be converted to a carboxylate side chain, and wherein the peptide has chromium binding activity. For example, a composition comprising an effective amount of a peptide comprising or consisting of SEQ ID NO: 3 (EEEEGDD) can be administered to the subject. This method can further comprise administering an effective amount of chromium to the subject. For example, a chromium(III) complex represented by the formula [Cr₃O(O₂CCH₂CH₃)₆(H₂O)³]⁺ can be administered to the subject (See U.S. Pat. No. 6,444,381). This method can further comprise administering an effective amount of insulin to the subject. This method can optionally comprise the step of diagnosing the subject with diabetes. Since the peptides disclosed herein increases insulin sensitivity, increases glucose uptake and/or increases insulin signaling in the subject, the amount of insulin necessary to achieve a therapeutic effect can be decreased. Therefore, when the peptide is administered in combination with insulin, the effective amount of insulin administered to the subject is lower than the diabetic dosage of insulin administered to the subject in the absence of treatment with the peptide. For example, the diabetic dosage of insulin can be decreased by 10%, 20%, 30%, 40%, 50%, 60%, 70%, 80%, 90% or 95% as compared to the dosage of insulin administered to the subject in the absence of treatment with the peptide. A decrease in the amount of insulin administered to the subject should be accompanied by a decrease in unwanted side effects. Insulin and/or chromium can be administered to the subject concurrently with a peptide disclosed herein. Insulin and/or chromium can be administered before or after administration of a peptide disclosed herein.

Further provided herein is a method of treating diabetes in a subject in need thereof, comprising administering a composition comprising an effective amount of a peptide disclosed herein, for example, a peptide comprising or consisting of SEQ ID NO: 3 (EEEEGDD) and an effective amount of a non-insulin therapeutic agent to the subject. This method can further comprise administering an effective amount of chromium to the subject. This method can optionally comprise the step of diagnosing the subject with diabetes.

Non-insulin therapeutic agents include, but are not limited to, biguanines, sulfonylureas, meglitinides, thiazolidinediones, dipeptidyl peptidase-4 inhibitors and glucagon-like peptide-1 receptor agonists. Since the peptides disclosed herein increase insulin sensitivity, increase glucose uptake and/or increase insulin signaling in the subject, the amount of a non-insulin therapeutic agent necessary to achieve a therapeutic effect can be decreased. Any of the therapeutic agents set forth herein can be administered with an anti-hyperlipidemic agent.

As used throughout, by subject is meant an individual. Preferably, the subject is a mammal such as a primate, and, more preferably, a human. Non-human primates include marmosets, monkeys, chimpanzees, gorillas, orangutans, and gibbons, to name a few. The term subject includes domesticated animals, such as cats, dogs, etc., livestock (for example, cattle, horses, pigs, sheep, goats, etc.) laboratory animals (for example, ferret, chinchilla, mouse, rabbit, rat, gerbil, guinea pig, etc.) and avian species (for example, chickens, turkeys, ducks, pheasants, pigeons, doves, parrots, cockatoos, geese, etc.). The subjects of the present invention can also include, but are not limited to amphibians and reptiles. Veterinary uses and formulations for same are also contemplated herein.

By “treat,” “treating,” or “treatment” is meant a method of reducing diabetes. Treatment can also refer to a method of reducing the disease or condition associated with diabetes rather than just the symptoms. The treatment can be any reduction from native levels and can be, but is not limited to, the complete ablation of the disease or the symptoms of the disease. Treatment can range from a positive change in a symptom or symptoms to complete amelioration as detected by art-known techniques. For example, a disclosed method is considered to be a treatment if there is about a 10% reduction in diabetes in a subject when compared to native levels in the same subject or control subjects. Thus, the reduction can be about a 10, 20, 30, 40, 50, 60, 70, 80, 90, 100%, or any amount of reduction in between as compared to native or control levels.

The peptides disclosed herein can also be used to prevent insulin sensitivity in a subject in need thereof.

The peptides set forth herein can be made by chemical synthesis methods that are well known to the ordinarily skilled artisan. See, for example, Fields et al., Chapter 3 in Synthetic Peptides: A User's Guide, ed. Grant, W.H. Freeman & Co., New York, N.Y., 1992. Peptides can be synthesized using the automated Merrifield techniques of solid phase synthesis with the alpha-NH₂ protected by either t-Boc or Fmoc chemistry using side chain protected amino acids on, for example, an Applied Biosystems Peptide Synthesizer Model 430A or 431. After complete assembly of the desired peptide, the resin is treated according to standard procedures to cleave the peptide from the resin and deblock the functional groups on the amino acid side chains. The free peptide is purified, for example by HPLC, and characterized biochemically, for example, by amino acid analysis, mass spectrometry, and/or by sequencing. Purification and characterization methods for peptides are well known to those of ordinary skill in the art. The peptide can also be produced by recombinant methods known to those of skill in the art.

The peptides and other therapeutic agents described herein can be provided in a pharmaceutical composition. Depending on the intended mode of administration, the pharmaceutical composition can be in the form of solid, semi-solid or liquid dosage forms, such as, for example, tablets, suppositories, pills, capsules, powders, liquids, or suspensions, preferably in unit dosage form suitable for single administration of a precise dosage. The compositions will include a therapeutically effective amount of the agent described herein or derivatives thereof in combination with a pharmaceutically acceptable carrier and, in addition, may include other medicinal agents, pharmaceutical agents, carriers, or diluents. By pharmaceutically acceptable is meant a material that is not biologically or otherwise undesirable, which can be administered to an individual along with the selected agent without causing unacceptable biological effects or interacting in a deleterious manner with the other components of the pharmaceutical composition in which it is contained.

As used herein, the term carrier encompasses any excipient, diluent, filler, salt, buffer, stabilizer, solubilizer, lipid, stabilizer, or other material well known in the art for use in pharmaceutical formulations. The choice of a carrier for use in a composition will depend upon the intended route of administration for the composition. The preparation of pharmaceutically acceptable carriers and formulations containing these materials is described in, e.g., Remington's Pharmaceutical Sciences, 21st Edition, ed. University of the Sciences in Philadelphia, Lippincott, Williams & Wilkins, Philadelphia Pa., 2005. Examples of physiologically acceptable carriers include buffers such as phosphate buffers, citrate buffer, and buffers with other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN® (ICI, Inc.; Bridgewater, N.J.), polyethylene glycol (PEG), and PLURONICS™ (BASF; Florham Park, N.J.).

Compositions containing the agent(s) described herein suitable for parenteral injection may comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents or vehicles include water, ethanol, polyols (propyleneglycol, polyethyleneglycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions and by the use of surfactants.

These compositions may also contain adjuvants such as preserving, wetting, emulsifying, and dispensing agents. Prevention of the action of microorganisms can be promoted by various antibacterial and antifungal agents, for example, parabens, chlorobutanol, phenol, sorbic acid, and the like. Isotonic agents, for example, sugars, sodium chloride, and the like may also be included. Prolonged absorption of the injectable pharmaceutical form can be brought about by the use of agents delaying absorption, for example, aluminum monostearate and gelatin.

Solid dosage forms for oral administration of the compounds described herein or derivatives thereof include capsules, tablets, pills, powders, and granules. The peptides disclosed herein can be derivatized with polyethylene glycol and other groups for oral administration. In such solid dosage forms, the compounds described herein or derivatives thereof is admixed with at least one inert customary excipient (or carrier) such as sodium citrate or dicalcium phosphate or (a) fillers or extenders, as for example, starches, lactose, sucrose, glucose, mannitol, and silicic acid, (b) binders, as for example, carboxymethylcellulose, alignates, gelatin, polyvinylpyrrolidone, sucrose, and acacia, (c) humectants, as for example, glycerol, (d) disintegrating agents, as for example, agar-agar, calcium carbonate, potato or tapioca starch, alginic acid, certain complex silicates, and sodium carbonate, (e) solution retarders, as for example, paraffin, (f) absorption accelerators, as for example, quaternary ammonium compounds, (g) wetting agents, as for example, cetyl alcohol, and glycerol monostearate, (h) adsorbents, as for example, kaolin and bentonite, and (i) lubricants, as for example, talc, calcium stearate, magnesium stearate, solid polyethylene glycols, sodium lauryl sulfate, or mixtures thereof. In the case of capsules, tablets, and pills, the dosage forms may also comprise buffering agents.

Solid compositions of a similar type may also be employed as fillers in soft and hard-filled gelatin capsules using such excipients as lactose or milk sugar as well as high molecular weight polyethyleneglycols, and the like.

Solid dosage forms such as tablets, dragees, capsules, pills, and granules can be prepared with coatings and shells, such as enteric coatings and others known in the art. They may contain opacifying agents and can also be of such composition that they release the active compound or compounds in a certain part of the intestinal tract in a delayed manner. Examples of embedding compositions that can be used are polymeric substances and waxes. The active compounds can also be in micro-encapsulated form, if appropriate, with one or more of the above-mentioned excipients.

Liquid dosage forms for oral administration of the compounds described herein or derivatives thereof include pharmaceutically acceptable emulsions, solutions, suspensions, syrups, and elixirs. In addition to the active compounds, the liquid dosage forms may contain inert diluents commonly used in the art, such as water or other solvents, solubilizing agents, and emulsifiers, as for example, ethyl alcohol, isopropyl alcohol, ethyl carbonate, ethyl acetate, benzyl alcohol, benzyl benzoate, propyleneglycol, 1,3-butyleneglycol, dimethylformamide, oils, in particular, cottonseed oil, groundnut oil, corn germ oil, olive oil, castor oil, sesame oil, glycerol, tetrahydrofurfuryl alcohol, polyethyleneglycols, and fatty acid esters of sorbitan, or mixtures of these substances, and the like.

Besides such inert diluents, the composition can also include additional agents, such as wetting, emulsifying, suspending, sweetening, flavoring, or perfuming agents.

Administration can be carried out using therapeutically effective amounts of the agents described herein for periods of time effective to increase insulin sensitivity or treat diabetes. The effective amount may be determined by one of ordinary skill in the art and includes exemplary dosage amounts for a mammal of from about 0.5 to about 200 mg/kg of body weight of active compound per day, which may be administered in a single dose or in the form of individual divided doses, such as from 1 to 4 times per day. Alternatively, the dosage amount can be from about 0.5 to about 150 mg/kg of body weight of active compound per day, about 0.5 to 100 mg/kg of body weight of active compound per day, about 0.5 to about 75 mg/kg of body weight of active compound per day, about 0.5 to about 50 mg/kg of body weight of active compound per day, about 0.5 to about 25 mg/kg of body weight of active compound per day, about 1 to about 20 mg/kg of body weight of active compound per day, about 1 to about 10 mg/kg of body weight of active compound per day, about 20 mg/kg of body weight of active compound per day, about 10 mg/kg of body weight of active compound per day, or about 5 mg/kg of body weight of active compound per day.

According to the methods taught herein, the subject is administered an effective amount of the agent. The terms effective amount and effective dosage are used interchangeably. The term effective amount is defined as any amount necessary to produce a desired physiologic response. Effective amounts and schedules for administering the agent may be determined empirically, and making such determinations is within the skill in the art. The dosage ranges for administration are those large enough to produce the desired effect in which one or more symptoms of the disease or disorder are affected (e.g., reduced or delayed). The dosage should not be so large as to cause substantial adverse side effects, such as unwanted cross-reactions, anaphylactic reactions, and the like. Generally, the dosage will vary with the activity of the specific compound employed, the metabolic stability and length of action of that compound, the species, age, body weight, general health, sex and diet of the subject, the mode and time of administration, rate of excretion, drug combination, and severity of the particular condition and can be determined by one of skill in the art. The dosage can be adjusted by the individual physician in the event of any contraindications. Dosages can vary, and can be administered in one or more dose administrations daily, for one or several days. Guidance can be found in the literature for appropriate dosages for given classes of pharmaceutical products.

Any appropriate route of administration may be employed, for example, parenteral, intravenous, subcutaneous, intramuscular, intraventricular, intracorporeal, intraperitoneal, rectal, or oral administration. Administration can be systemic or local. Multiple administrations and/or dosages can also be used. Effective doses can be extrapolated from dose-response curves derived from in vitro or animal model test systems.

Nucleic acids encoding the peptides disclosed herein can also be employed. The nucleic acid can be delivered intracellularly (for example by expression from a nucleic acid vector or by receptor-mediated mechanisms), or by an appropriate nucleic acid expression vector which is administered so that it becomes intracellular, for example by use of a retroviral vector (see U.S. Pat. No. 4,980,286), or by direct injection, or by use of microparticle bombardment (such as a gene gun; Biolistic, Dupont), or coating with lipids or cell-surface receptors or transfecting agents.

Physical transduction techniques can also be used, such as liposome delivery and receptor-mediated and other endocytosis mechanisms (see, for example, Schwartzenberger et al., Blood 87:472-478, 1996) to name a few examples. These methods can be used in conjunction with any of these or other commonly used gene transfer methods.

### Composition

Provided herein is a synergistic pharmaceutical combination comprising a first pharmaceutical composition comprising an anti-diabetic agent and second pharmaceutical composition comprising a peptide, wherein the peptide comprises 10 amino acids or less in length, wherein about 50% of the amino acids or greater are amino acids with a carboxylate side chain or amino acids with a side chain that can be converted to a carboxylate side chain, and wherein the peptide has chromium binding activity. For example, and not to be limiting, this can be a peptide comprising or consisting of SEQ ID NO: 3. Thus, provided herein is the use of a peptide disclosed herein for the preparation of a pharmaceutical composition which synergistically enhances the effect of an anti-diabetic agent. The anti-diabetic agent can be, for example, insulin, a biguanine, a sulfonylurea, a meglitinide, a thiazolidinedione, a dipeptidyl peptidase-4 inhibitor or a glucagon-like peptide-1 receptor agonist, to name a few. In the pharmaceutical combination disclosed herein, the effective dosage of the anti-diabetic agent used in combination with the peptide is less than the effective dosage of the anti-diabetic agent when used alone. For example, the effective dosage of the anti-diabetic agent can be about 10%, 20%, 30%, 40%, 50%, 60%, 70%, 80%, 90% less than the effective dosage of the diabetic agent when used alone.

A pharmaceutical combination is an association of two pharmaceutically active agents in which 1) each of the active agents has been converted to separate pharmaceutical compositions using one or more conventional carrier(s) and any of the usual processes of drug manufacture or 2) the two active agents have been converted to one single pharmaceutical composition that can be administered to the patient being in need thereof. In the latter case, the pharmaceutical composition may contain a mixture of the two active agents, or each of the active agents may be present at a different site in the pharmaceutical composition, e.g. one of them in the tablet core and the other in a coating of the tablet core. It is understood that one or more conventional carriers and any of the usual processes of drug manufacture can be used to prepare this single pharmaceutical composition.

The pharmaceutical combination comprising the anti-diabetic agent can be administered to the subject simultaneously with, before or after administration of the pharmaceutical composition comprising the peptide. The pharmaceutical combination can also be administered with another non-diabetic therapeutic agent, for example, an anti-hyperlipidemic agent.

In this pharmaceutical combination, the anti-diabetic agent and the peptide can be in separate formulations. The formulations can be in unit dosage formulations. The peptide formulation can further comprise chromium.

A number of aspects have been described. Nevertheless, it will be understood that various modifications may be made. Furthermore, when one characteristic or step is described it can be combined with any other characteristic or step herein even if the combination is not explicitly stated. Accordingly, other aspects are within the scope of the claims.

The following examples are put forth so as to provide those of ordinary skill in the art with a complete disclosure and description of how the compounds and/or methods claimed herein are made and evaluated, and are intended to be purely exemplary of the invention and are not intended to limit the scope of what the inventors regard as their invention except as and to the extent that they are included in the accompanying claims.

### EXAMPLES

Chromium has been proposed to be an essential element over fifty years ago and has been shown to have therapeutic potential in treating the symptoms of type 2 diabetes; however, its mechanism of action at a molecular level is unknown. As set forth herein, one chromium-binding biomolecule, low-molecular-weight chromium-binding substance (LMWCr or chromodulin), has been found to be biologically active in in vitro assays and is likely involved in the in vivo biologically active form of chromium. Characterization of the organic component of LMWCr has proven difficult. Treating bovine LMWCr with trifluoroacetic acid followed by purification on a graphite powder micro-column generates a heptapeptide fragment of LMWCr. The peptide sequence of the fragment was analyzed by mass spectrometry (MS) and tandem MS (MS/MS and MS/MS/MS) using collision-induced dissociation (CID) and post-source decay (PSD). Two candidate sequences, pEEEEGDD (SEQ ID NO:1) and pEEEGEDD (SEQ ID NO: 2) (where pE is pyroglutamate), were identified from MS/MS experiments; additional tandem mass spectrometry suggests the sequence is pEEEEGDD (SEQ ID NO: 1). The N-terminal glutamate residues explain the inability to sequence LMWCr by the Edman method. Langmuir isotherms and Hill plots were used to analyze the binding constants of chromic ions to synthetic peptides similar in composition to apoLMWCr. The sequence pEEEEGDD (SEQ ID NO: 1) was found to bind four chromic ions per peptide with nearly identical cooperativity and binding constants to those of apoLMWCr.

Despite chromium being proposed as an essential trace element over fifty years and having been demonstrated to have potential as a adjuvant therapy to improve insulin resistance and related symptoms in rodent models of type 2 diabetes, the mode of action of chromium at a molecular level has not been elucidated. Two biomolecules are known to bind chromium: transferrin and low-molecular-weight chromium-binding substance. Prior to this disclosure, no amino acid sequence data was available for LMWCr, despite attempts at sequencing by Edman degradation, NMR, and mass spectrometry. Set forth herein are successful efforts to sequence the oligopeptide of LMWCr. Further provided is evidence that a synthetic peptide of this sequence binds Cr in a similar fashion to LMWCr and increases insulin-stimulated glucose uptake in vitro and in vivo.

### Experimental Procedures

### Materials

α-Cyano-4-hydroxycinnamic acid (CHCA), 2,5-dihydroxybenzoic acid (DHB), trifluoroacetic acid (TFA) and activated charcoal (C-5510) were obtained from Sigma (St. Louis, Mo.). LC-MS grade acetonitrile was obtained from Riedel-de Haën (Seelze, Germany). LMWCr were purified from livers of alligator (Hatfield et al. “Low-molecular-weight Chromium-binding Substance from Chicken Liver and American Alligator Liver,” Comp Biochem Physiol Part B. 2006; 144:423-431), bovine (Davis et al. “Isolation and characterization of a biologically active form of chromium oligopeptide from bovine liver,” Arch Biochem Biophys. 1997; 339:335-343), chicken (Hatfield et al.) and human urine (Chen Y. “Low-molecular-weight chromium-binding substance: Advanced studies from ayes to human,” Ph.D. dissertation, The University of Alabama, 2009) utilizing methods described previously. The peptides pEEEEGDD (SEQ ID NO: 1) and pEEEGEDD (SEQ ID NO: 2) were synthesized using standard Fmoc procedures (Chan WC and White. Fmoc Solid Phase Peptide Synthesis: A Practical Approach. New York: Oxford University Press; 2000, p. 345) with an Advanced ChemTech Model 90 peptide synthesizer. ⁵¹CrCl₃ was obtained from ICN (Irvine, Calif.); CrCl₃ was obtained from Fisher Scientific. Hepes was obtained from Research Organics, Inc (Cleveland, Ohio).

### Mass Spectrometry

Matrix-assisted laser desorption ionization time-of-flight mass spectrometry (MALDI/TOF MS) was performed on a Bruker Daltonics Reflex III mass spectrometer with a two stage reflectron (Clipston et al. “A comparison of negative and positive ion time-of-flight post-source decay mass spectrometry for peptides containing basic residues,” Int J Mass Spectrom 2003; 222:363-381). Ionization used the 337 line of a Laser Science (Franklin, Mass., USA) VSL-337ND-S nitrogen laser. Positive and negative ion spectra were obtained in linear and reflectron modes with an accelerating voltage of 20 kV. Post-source decay (PSD) spectra used precursor ion selection with a pulsed voltage that deflected matrix and contaminant ions from entering the flight tube. Product ions were detected in segments by stepping down the reflectron voltage as follows: −21.0, −19.55, −15.75, −11.82, −8.86, −6.64, −4.98, −3.74, and −2.80 kV. The MALDI matrix was generally α-cyano-4-hydroxycinnamic acid (CHCA), although some experiments utilized 2,5-dihydroxybenzoic acid (DHB).

For LC-MS analysis, an Agilent 1200 series liquid chromatograph with a Zorbax (150×0.5 mm) 5B-C18 column was interfaced to a Bruker HCT ultra PTM discovery system high capacity quadrupole ion trap (QIT) mass spectrometer via electrospray ionization (ESI). The mobile phase involved doubly deionized water (ddH₂O) and acetonitrile; gradient elution was employed. Direct infusion experiments used a syringe pump with a flow rate of approximately 140 μL/h. The ESI needle spray voltage was 4 kV; the capillary temperature was 300° C.; and mass spectra were acquired over a range of m/z 100-2000. Low energy collision-induced dissociation (CID) used helium as the collision gas. The fragmentation amplitude was 1.0 V, and the acquisition software's smart fragmentation was on (the start Amplitude 30% and the end amplitude 200%).

### Graphite Powder Microcolumn

Custom-made chromatographic microcolumns were used for desalting and concentration of the peptide prior to MS analysis. Activated charcoal was packed in a constricted gel loader tip (Eppendorf). A 10-mL syringe was used to force liquid through the column by applying gentle air pressure. The columns were equilibrated with 10 μL of 0.1% TFA. An aliquot of the LMWCr after purification by Sephadex G-15 column chromatography was diluted to 30 μL in 0.1% TFA and loaded onto the column using gentle syringe air pressure. The column was washed with 60 μL 0.1% TFA. The resulting samples were mixed with approximately 2 μL of 4HCCA in 70% acetonitrile/0.1% TFA and spotted onto the MALDI target (plate) with a micropipettor.

In order to generate more purified samples, ESI/MS (electrospray ionization mass spectrometry) samples were prepared with a modified method in which activated charcoal powder was packed into a microconcentrator instead of the gel loader tip. A tabletop centrifuge was used to wash the sample and elute the sample through charcoal powder and filter membrane. A solvent of 70% acetonitrile/0.1% TFA was used for the final elution solution. Eluent was lyophilized and re-dissolved in ddH₂O before LC-MS processing.

### Chromium Binding Studies

A variation of the equilibrium dialysis method using an ultrafiltration device was utilized to examine the binding of chromium to the synthetic peptides. Aliquots of a mixture of CrCl₃ and ⁵¹CrCl₃ were combined to generate different concentrations of Cr(III) while maintaining the synthetic peptide in solution at a constant volume. Known amounts (approximately 0.46 μmol) of peptide and 200 mL of 0.1 mol/L Hepes buffer (pH 7.4) were slowly stirred in an Amicon 8400 ultrafiltration unit (with a YC05 membrane) at 4° C. temperature for at least 12 h to achieve equilibration. The ultrafiltration unit was then pressurized, and effluent was collected. The content of free chromic ion in the effluent was determined by gamma counting using a Packard Cobra II auto-gamma counter. Chromium binding experiments were performed in triplicate, and the synthetic peptide used in at least one of the three sets of triplicates was from a different synthesis. As a control for the chromium-binding experiments, the ultrafiltration procedure was performed without peptide to establish the amount of chromium that adhered to the ultrafiltration unit; all experiments were corrected for this background. Linear regression analyses of the Langmuir isotherms and Hill plots were performed using SigmaPlot 11.0. The Cr concentration of solutions of isolated peptides were determined by graphite furnace atomic absorption spectroscopy using a PerkinElmer Analyst 400 atomic absorption spectrometer equipped with an HGA-900 graphite furnace and an AS-800 autosampler using a chromium hollow cathode lamp operating at 10 mA; a spectral bandwidth of 0.8 nm was selected to isolate the light at 353.7 nm. Chromium standard solution obtained from Perkin Elmer (Waltham, Mass.) was utilized to generate a standard curve.

Errors are presented throughout as standard deviations of the triplicate analyses. Doubly deionized H₂O was used throughout. Amino acid analyses of samples were performed by the Protein and Separation Analysis Laboratory at Purdue University. Protein concentrations were determined by the fluorescamine method (Davis et al. “Isolation and characterization of a biologically active form of chromium oligopeptide from bovine liver,” Arch Biochem Biophys. 1997; 339:335-343.) Fluorescence measurements were obtained on a Jobin Yvon FluoroMax-3 fluorescence spectrophotometer. Ultraviolet-visible spectra were obtained using a Hewlett-Packard 8451A or a Beckman Coulter DU 800 spectrophotometer.

### Results

### Production of Apo-Oligopeptide of LMWCr

Treatment of LMWCr from a variety of sources (alligator liver, chicken liver, bovine liver, and human urine) with 0.1% TFA resulted in no visible precipitation. Separation of the products was attempted by GP microcolumn. Graphite powder (GP) has been utilized to effectively retain small and hydrophilic peptides, which could readily be eluted for mass spectral analysis. The primary organic product eluting from the column contained no detectable chromium by the diphenylcarbazide method. Attempts to assay for protein content via reactions with a free primary amine group with fluorescamine indicated that the isolated material was either not a protein or that the amino terminus was blocked. Amino acid analysis of the component of bovine LMWCr eluting from the graphite powder (GP) column produced the composition 1.0 glycine: 4.5 glutamate (and/or glutamine): 2.2 aspartate (and/or asparagine): 0 cysteine, indicating that the LMWCr had lost some of its amino acids during the TFA treatment and/or GP microcolumn processing. No other amino acids were detected above trace quantities. If the amino acid ratio is calculated assuming 2.0 aspartate residues, then the ratio becomes 0.91 glycine: 4.1 glutamate: 2.0 aspartate, indicating the loss of one glycine residue and two cysteine residues compared to the original composition of bovine LMWCr (2 glycine:4 glutamate: 2 aspartate: 2 cysteine).

### MALDI/TOF MS Studies

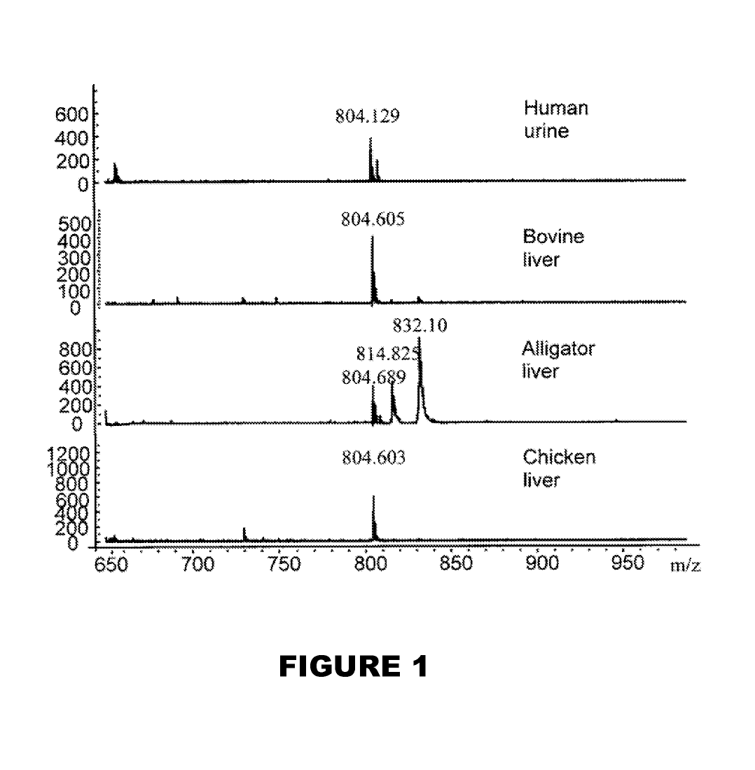

A molecular ion (m/z 804) was observed for all treated LMWCr samples with GP column under negative mode MALDI/TOF MS (FIG. 1). No corresponding m/z peak was found under positive mode. The lack of a positive signal, [M+H]⁺, is consistent with the highly acidic nature of LMWCr. The intensities for the ions of interest are only a few hundred detector counts, which is very low; a more typical value would be about ten times higher. The low signal intensity possibly resulted from poor binding capacity of the microcolumn or inefficient elution using 70% acetonitrile/0.1% TFA, or the samples not being ionized well.

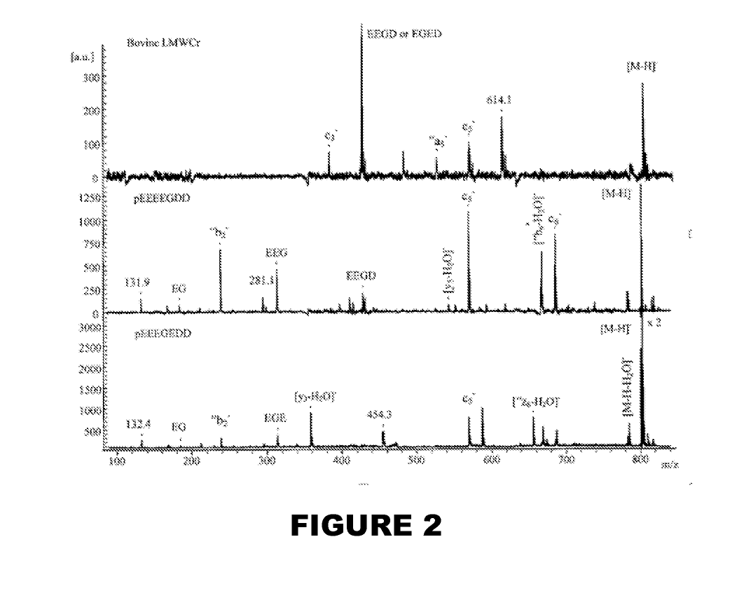

Post source decay (PSD) of the m/z 804 ion of bovine LMWCr was performed (FIG. 2), and two sequences were proposed based on this data: pEEEEGDD (SEQ ID NO:1) and pEEEGEDD (SEQ ID NO: 2) (where pE is pyroglutamate). Peptide backbone cleavage ions were identified and are denoted in FIG. 2 with Roepstorffand Fohlman nomenclature. All assigned product ions match the mass-to-charge (m/z) of the predicted ions to within m/z ±1, which is within accepted accuracy of PSD. There are several unassigned peaks of appreciable intensity in the spectra that are not standard peptide cleavage fragments. The precursor ion, [M−H]⁻, at m/z 804 is 2 Da higher in mass than expected.

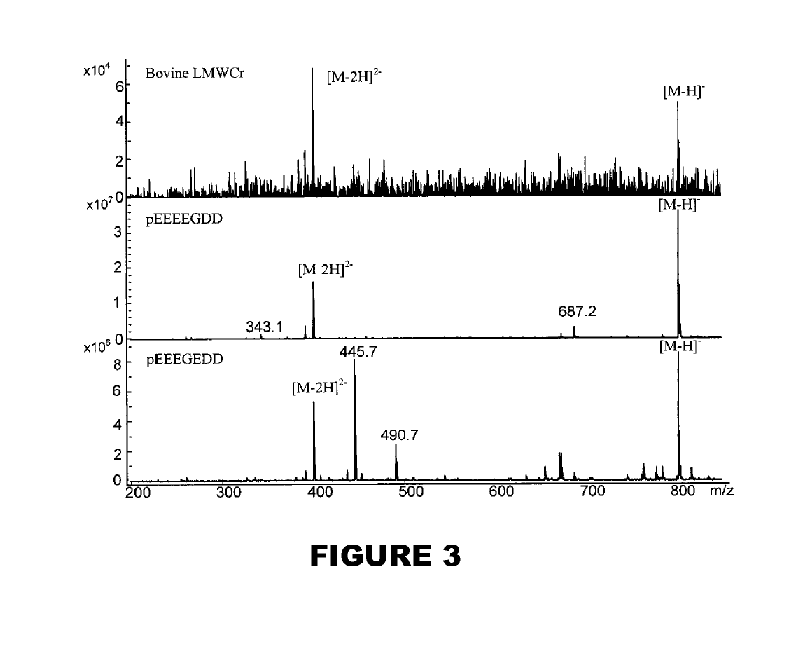

Post-source decay spectra of synthetic peptides were generated and compared to those of the LMWCr's; the spectra are shown in FIG. 3. The synthetic peptides produced the expected [M−H]⁻ at m/z 802. The PSD spectra for the biological LMWCr's were produced from m/z 804, while the PSD spectra for the synthetic peptides were generated from m/z 802. (MALDI/TOF MS analysis of mixtures of biological and synthetic peptides yielded both m/z 802 and m/z 804, showing that these were distinct ions.) This mass discrepancy may prevent a strong match between the PSD spectra for the biological peptides and the model peptides. The spectra of the LMWCr's from different biological sources shared common features at m/z 384, 428, 482, and 570, which suggests a similarity in sequence. However, the PSD spectra of peptides pEEEEGDD (SEQ ID NO: 1) and pEEEGEDD (SEQ ID NO: 2) both showed only a few similar features at m/z 428, 482, and 570 to those of the LMWCr's. Neither is a sufficient match to positively identify the biological peptide.

Analysis of LMWCr using ESI/QIT MS

Larger samples of LMWCr were isolated by the modified GP column with the application of the microconcentrator. The ability of reverse phase column (Zorbax 5B-C18 on LC/MS) to retain LMWCr was tested; experiments revealed that the products of the TFA treatment of LMWCr elute during the first 5 min when washing column with 2% acetonitrile; these were detected by obvious UV absorbances at 260 nm. No ESI response (m/z 802) could be observed because ionization interferences occur when an extract from a biological specimen, LMWCr in this case, is loaded into the LC portion of the instrument. Suppression of the signal at the time point that corresponds to the void volume of the column is common. Consequently, for the experiments described below, LMWCr samples were introduced into the ESI source by infusion with a syringe pump rather than by LC.

As was the case for MALDI, no positive ion signal was observed when LMWCr samples were ionized by ESI. The negative mode ESI spectrum of bovine LMWCr (FIG. 3) shows one peak at m/z 802 and another at m/z 401 corresponding to the singly and doubly charged species, [M−H]⁻ and [M−2H]²⁻, respectively. Unlike the ions generated by MALDI, these ions generated by ESI (which is a much gentler ionization technique) exactly conform to the MW of pEEEEGDD (SEQ ID NO: 1) or pEEEGEDD (SEQ ID NO: 2). Low-energy CID MS/MS (MS²) on m/z 802 ions, [M−H]⁻, was carried out to elucidate the sequence. The synthetic peptides pEEEEGDD (SEQ ID NO: 1) and pEEEGEDD (SEQ ID NO: 2) were dissociated under the same conditions. The MS/MS spectra of m/z 802, [M−H]⁻, from the bovine sample and the two synthetic peptides were dominated by a very intense water elimination ion at m/z 784, [M−H−H₂O]⁻. Water loss during low-energy CID is common and abundant in negative mode when a peptide has adjacent acidic residues. Relative to this large m/z 784, other CID products were only a few percent relative intensity (or less), and no obvious differences existed among these low intensity ions. That is, CID on [M−H]⁻ (MS/MS) cannot distinguish between these two synthetic peptides, and both model spectra are a good match for the biological sample.

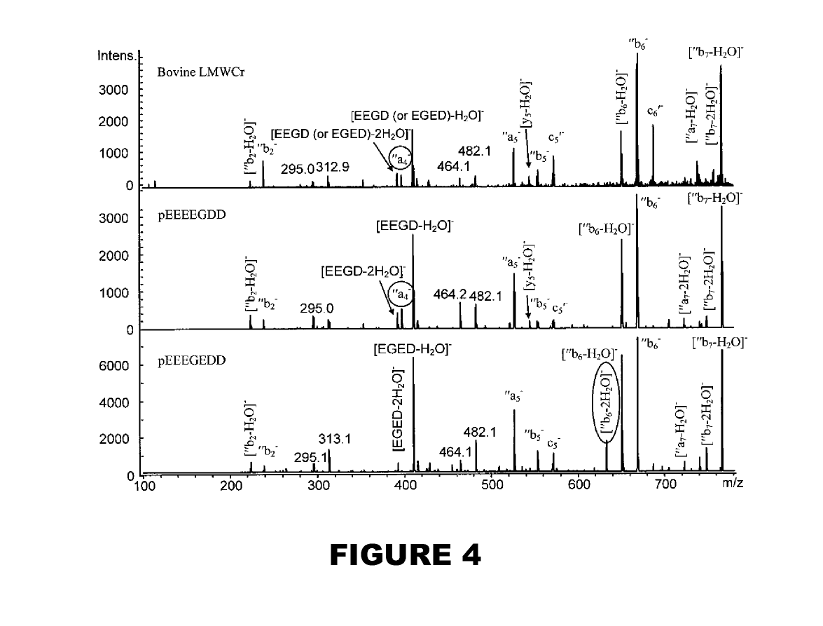

The intense peak at m/z 784, [M−H−H₂O]⁻, which dominated the MS/MS spectra for both synthetic peptides and for bovine LMWCr, was subjected to a further stage of CID. The resulting MS/MS/MS (or MS³) spectra are shown in FIG. 4. Again very similar spectral features were shared by LMWCr and the two synthetic peptides. A few notable differences were observed in MS/MS/MS spectra of m/z 784 ions between the two synthetic peptides. A peak at m/z 397 (circle marked in FIG. 4) is found in the spectrum from pEEEEGDD (SEQ ID NO: 1); this corresponds to an ″a₄⁻. Because ″a₄⁻ incorporates only the first four residues of the peptides (starting at the N-terminus), it will not form at the same m/z in the spectrum of pEEEGEDD (SEQ ID NO: 2). The CID spectrum from the fragment of bovine LMWCr also contains a peak at m/z 397 in roughly the same abundance as in the spectrum for pEEEEGDD (SEQ ID NO: 1). In addition, a peak at m/z 632, corresponding to [″b₆-2H₂O]⁻, is found only in the spectra from pEEEGEDD (SEQ ID NO: 2), but not the spectra from pEEEEGDD (SEQ ID NO: 1) or bovine LMWCr. In negative mode CID of peptides, adjacent acidic residues (aspartic acid or gluatmic acid) promote water loss, and this is much more prevalent when one of the residues is aspartic acid. Of the two model peptides, only pEEEGEDD (SEQ ID NO: 2) has an aspartic acid residue (D at the sixth position) adjacent to another acidic residue (E at the fifth position) within the first six residues of the sequence, which comprise [″b₆-2H₂O]⁻. Taking into account the two spectral features discussed here, the sequence of the fragment from bovine LMWCr is assigned as pEEEEGDD (SEQ ID NO: 1).

Bioinformatics

In the body, peptides, including numerous peptides with bioactivity, originate from the processing of proteins. Thus, the heptapeptide isolated from LMWCr should have at a point in its history been be part of a larger protein. A genomic search against the databases of the National Center for Biotechnology Information (NCBI) using the sequence EEEEGDD (SEQ ID NO: 3) was performed to identify proteins containing this sequence motif. Multiple 100% hits were found due to the short sequence and low complexity: seven sequences in Homo sapiens; two in Bos taurus; two in Gallus gallus; one in Mus musculus. Unfortunately, very little of the American alligator genome has been sequenced. None of the hits contain glycine and cysteine residues flanking the EEEEGDD (SEQ ID NO: 3) sequence, suggesting that these residues are not part of a contiguous peptide and are attached to the heptapeptide in a non-standard fashion.

### Chromium-Binding

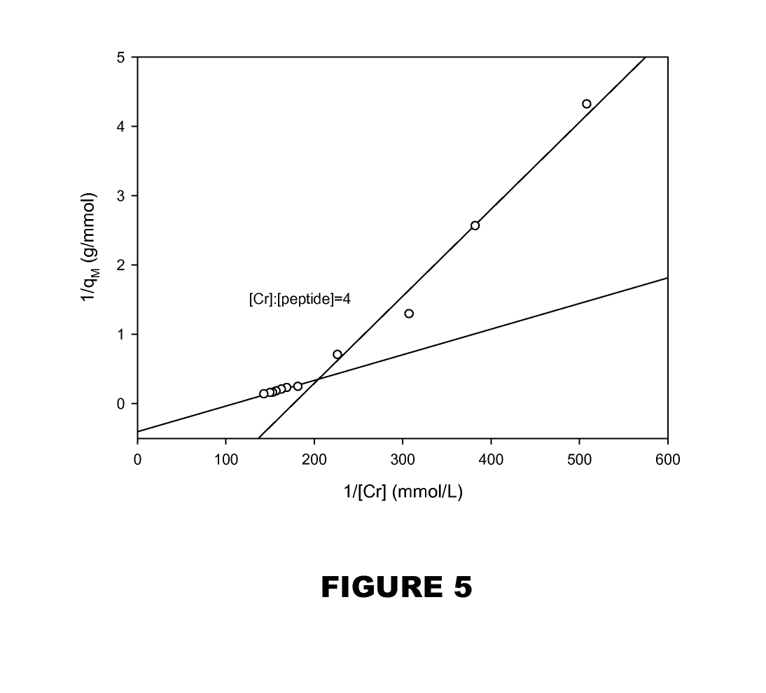

As chromium binding to the oligopeptide LMWCr is believed to be through only carboxylate residues and the heptapeptide pEEEEGDD (SEQ ID NO: 1) retains all the aspartate and glutamate residues in LMWCr, whether the heptapeptide can bind chromium in a similar fashion to LMWCr was investigated. The binding of chromium to bovine apoLMWCr prepared by the low pH EDTA method (Davis and Vincent), the heptapeptide pEEEEGDD (SEQ ID NO: 1), and two other acidic peptides was probed. The two peptides EDGEECDCGE(SEQ ID NO: 41) and DGEECDCGEE (SEQ ID NO: 42) were chosen as they possess the same amino acid composition as bovine LMWCr, and searches of the human genome reveal these to be the only two sequences in this genome with this composition. The number of Cr³⁺ ions binding to the peptides was estimated using Langmuir isotherms. For the Langmuir isotherms of Cr³⁺ binding to all the synthetic peptides, Cr binding to apoLMWCr can be represented by two intersecting straight lines with different slopes (FIG. 5)). This biphasic behavior indicates that each peptide has two types of Cr³⁺ binding sites: tight binding sites and weak binding sites. Negative values of the Langmuir parameters Bₜ and K were found for each peptide: apoLMWCr, −2.69 mmol/g and −109 L/mmol, respectively; EDGEECDCGE (SEQ ID NO: 41), −6.97 mmol/g and −354 L/mmol; DGEECDCGEE (SEQ ID NO: 42), −62.9 mmol/g and −9.94 L/mmol; and pEEEEGDD (SEQ ID NO: 1), −0.73 mmol/g and −151 L/mmol. The appearance of negative Bₜ values for all peptides demonstrates the limitation of using the simple Langmuir model in cases of tight-binding. The negative value of Bₜ indicates that most of the sorption sites have a high affinity for Cr³⁺ ions, especially at low Cr³⁺ concentrations.

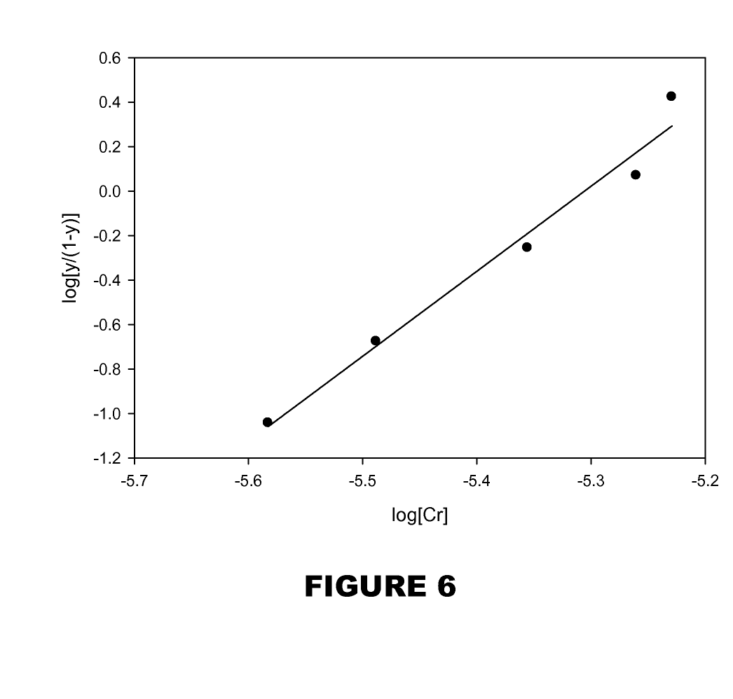

The intersection points in the isotherms allow the number of tightly binding ions to be estimated. For bovine apoLMWCr, the chromium:oligopeptide ratio at the intersection point is 3.6, which is very close to the average amount of chromium bound to isolated bovine LMWCr, which is 3.5. Titration of bovine LMWCr with Cr³⁺ has previously shown that four Cr³⁺ ions are required to restore bioactivity. For the synthetic peptides, the intersection points occurred at Cr:peptide ratios of approximately 2, 2, and 4 for EDGEECDCGE (SEQ ID NO: 41), DGEECDCGEE (SEQ ID NO: 42) and pEEEEGDD (SEQ ID NO: 1) (FIG. 6), respectively. This is consistent with the Crpeptides binding ratios found after exposing the peptides to solutions of 10 equivalents of Cr³⁺ (CrCl₃.6H₂O in 0.1 mol/L hepes buffer, pH 7.4 overnight at 4° C.) and separating the peptides from the excess Cr³⁺ by G-10 size exclusion chromatography (EDGEECDCGE (SEQ ID NO: 41): 2.0(±0.3), and DGEECDCGEE (SEQ ID NO: 42): 2.0±0.3). A change in mode from specific coordinate covalent binding to just electrostatic absorption on the peptide surface is proposed. Thus, the inflection point of the isotherm for the peptide pEEEEGDD (SEQ ID NO: 1) implies that it tightly binds four Cr³⁺ ions. The binding of two Cr³⁺ ions to the peptides EDGEECDCGE (SEQ ID NO: 41) and DGEECDCGEE (SEQ ID NO: 42) is consistent with mass spectrometric studies of chromium-binding to these peptides. The electronic spectrum of Cr-loaded pEEEEGDD (SEQ ID NO: 1) peptide has two visible maxima at ˜411 and 577 nm and a broad shoulder in the ultraviolet region at ˜270 nm. The two visible features are readily assigned to d→d transitions from the Cr³⁺ centers. The spectrum is very similar to that of bovine liver LMWCr (shoulder ˜260 nm, 394 nm, 576 nm).

To further compare the binding properties between the synthetic peptides and apoLMWCr in CrCl₃ solution, the method of Hill was applied to establish the degree of cooperativity between the covalent binding sites with the initial low amounts of substrate in solution, assuming covalent binding sites are occupied before surface adsorption occurs. The total number of tight binding sites established using the Langmuir isotherm was utilized. This method uses the binding number y defined as

$$y = \frac{{{K}_{f} [ Cr ]}^{n}}{1 + {{K}_{f} [ Cr ]}^{n}} \qquad Eqn . 1$$

where K_{f} is the binding of formation constant and n is the Hill constant such that

$$log [ \frac{y}{1 - y} ] = log {K}_{f} + n log [ Cr ] \qquad Eqn . 2$$

Hill plots gave linear curves (FIG. 6). K_{f} and n were obtained as the value of y-intercept and slope, respectively (Table 1). These data indicate a large degree of positive cooperativity such that the binding of the first and subsequent Cr³⁺ ions facilitates the binding of addition Cr, perhaps in a multinuclear assembly; the magnitudes suggest that essentially only apopeptide or peptide saturated with Cr³⁺ ions exist in solution. The Hill constants, K_{f} and n, of apoLMWCr measured in this study, 1.10×10²¹ (mol/L)⁻⁴ and 3.82, differ only slightly from published data, K_{f}=1.54×10²¹ (mol/L)⁻⁴ and n=3.47. For EDGEECDCGE (SEQ ID NO: 41), the Hill constant is greater than the number of interacting sites, as only two Cr(III) binding sites are on the peptide. This shows that the resulting Cr-peptide complex is actually a dimer of peptide. As both apoLMWCr and pEEEEGDD (SEQ ID NO: 1) bind four Cr³⁺ ions and the binding constants for apoLMWCr and pEEEEGDD (SEQ ID NO: 1) are within an order of magnitude while the Hill constants are identical, pEEEEGDD (SEQ ID NO: 1) appears to contain all the essential components of LMWCr for binding chromium and probably binds chromium in an essentially identical fashion to that of LMWCr.

TABLE 1
Hill plot constants K_{f} and n for chromium(III) binding to bovine
liver apoLMWCr and synthetic peptides.

| Peptide | K_{f} | n | Co-operativity |
|---|---|---|---|
| ApoLMWCr | 1.10 × 10²¹ (mol/L)⁻⁴ | 3.82 | Positively co-operative |
| (bovine liver) | | | |
| EDGEECDCGE | 1.48 × 10²³ (mol/L)⁻² | 3.64 | Positively co-operative |
| (SEQ ID NO: 41) | | | |
| DGEECDCGEE | 1.01 × 10¹¹ (mol/L)⁻² | 1.85 | Positively co-operative |
| (SEQ ID NO: 42) | | | |
| pEEEEGDD | 1.92 × 10²⁰ (mol/L)⁻⁴ | 3.82 | Positively co-operative |
| (SEQ D NO: 1) | | | |

### Glucose Uptake

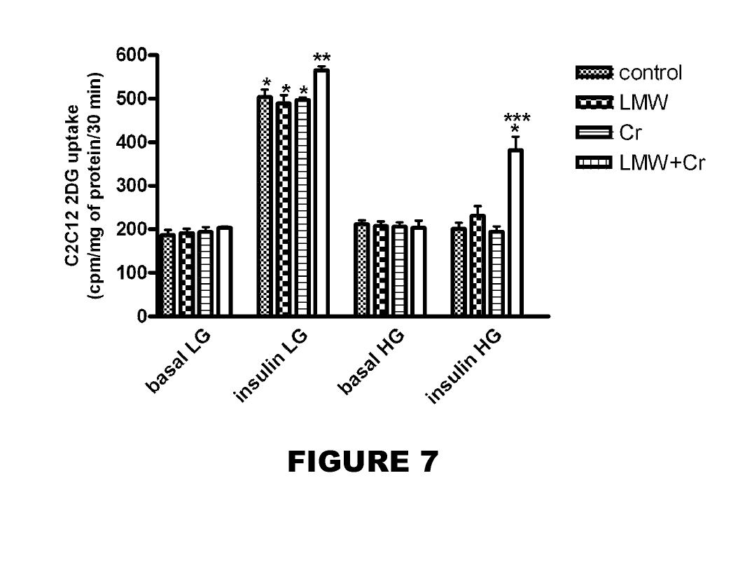

The endogenous chromium-binding peptide EEEEGDD (SEQ ID NO: 3) augments insulin-stimulated glucose uptake in cultured myotubes incubated overnight in 10 mM (low glucose) or 25 mM glucose (high glucose to induce insulin resistance) (FIG. 7). Myotubes were incubated with the peptide (10 mM) and/or chromium for 1 h and stimulated with insulin (50 nM) for 10 min, and 2-[³H]-deoxyglucose uptake was assessed. Data represent mean counts per minute (cpm)/mg protein/30 min±SE (n=3). *p<0.01 compared to basal low glucose conditions. **p<0.01 compared to insulin-stimulated low glucose conditions. ***p<0.001 compared to high glucose conditions.

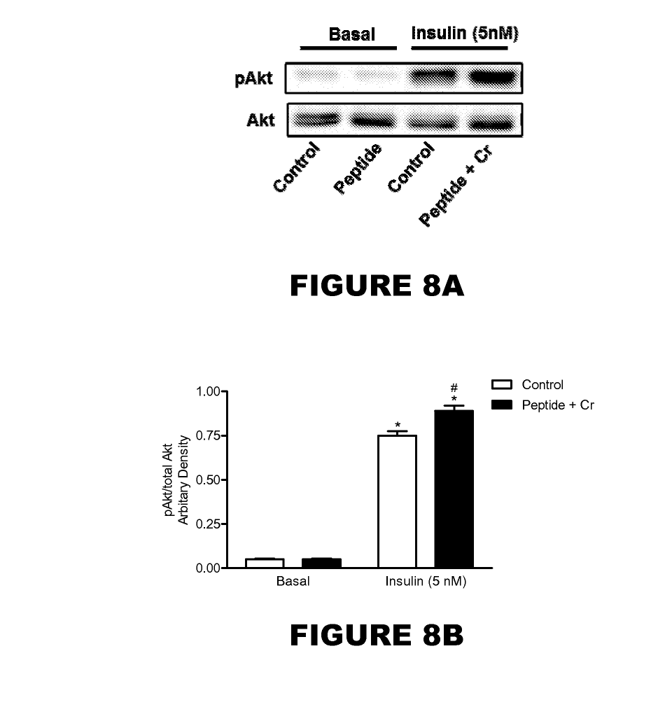

The endogenous chromium-binding peptide EEEEGDD (SEQ ID NO: 3) augments insulin-stimulated glucose uptake in vivo (FIG. 8). The peptide (5 μmol/kg) either alone or premixed with equimolar concentrations of chromium were injected via tail-vein, immediately following which glucose (2 g/kg body weight) was administered intraperitoneally (*p<0.05, n=6-8).

### Phosphorylation of Akt

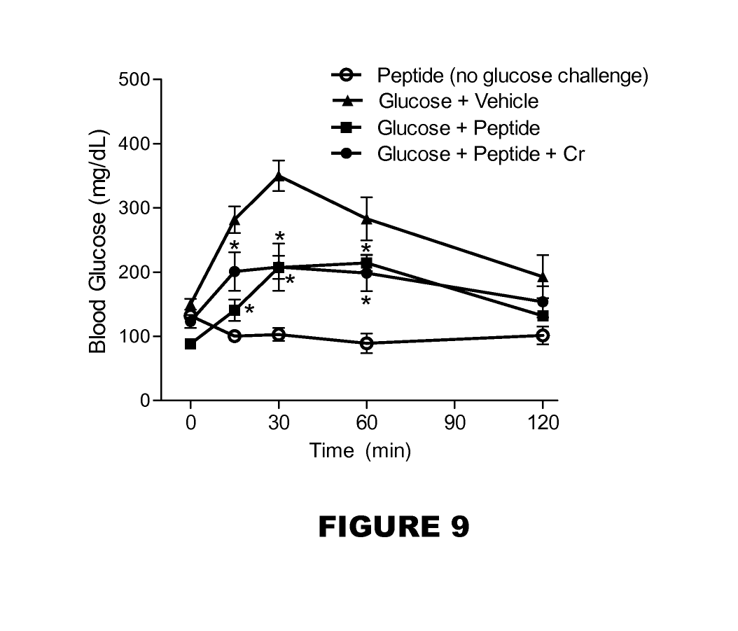

The endogenous chromium binding peptide EEEEGDD (SEQ ID NO: 3) improves insulin-stimulated phosphorylation of Akt in cultured myotubes (FIG. 9). Myotubes were incubated with the peptide and/or chromium (1 μM) for 1 h and stimulated with insulin 5 nm. (A) Representative Western blots of p³⁰⁸-Akt and Akt, and (B) Densitometric quantitation of the Western blots. Data represent mean±SE (n=3), *p<0.001 versus control **p<0.01 compared to insulin-stimulated glucose uptake (panel A). *p<0.001 versus control ^{#}p<0.05 compared to insulin-stimulated glucose uptake in untreated cells (panel B).

The compositions and methods of the appended claims are not limited in scope by the specific compositions and methods described herein, which are intended as illustrations of a few aspects of the claims and any compositions and methods that are functionally equivalent are within the scope of this disclosure. Various modifications of the compositions and methods in addition to those shown and described herein are intended to fall within the scope of the appended claims. Further, while only certain representative compositions, methods, and aspects of these compositions and methods are specifically described, other compositions and methods and combinations of various features of the compositions and methods are intended to fall within the scope of the appended claims, even if not specifically recited. Thus a combination of steps, elements, components, or constituents can be explicitly mentioned herein; however, all other combinations of steps, elements, components, and constituents are included, even though not explicitly stated.

What is claimed is:

1. A method for increasing insulin sensitivity in a subject in need thereof, comprising:
a) identifying a subject in need of increased insulin sensitivity; and b) administering to the subject a composition comprising an effective amount of a peptide, wherein the peptide comprises 10 amino acids or less in length, wherein about 50% of the amino acids or greater are amino acids with a carboxylate side chain or amino acids with a side chain that can be converted to a carboxylate side chain, and wherein the peptide has chromium binding activity.

2. The method of claim 1, wherein the peptide comprises an amino acid sequence according to the formula XXXXGXX (SEQ ID NO: 4), wherein X is an amino acid with a carboxylate side chain or an amino acid with a side chain that can be converted to a carboxylate side chain.

3. The method of claim 2, wherein the peptide is a peptide comprising SEQ ID NO: 3 (EEEEGDD).

4. The method of claim 3, wherein the peptide is a peptide consisting of SEQ ID NO: 3.

5. The method of claim 1, further comprising administering an effective amount of chromium to the subject.

6. The method of claim 1, wherein the subject has diabetes.

7. The method of claim 6, wherein the subject has Type 2 diabetes.

8. The method of claim 1, wherein the peptide does not comprise pyroglutamate.

9. A method of treating diabetes in a subject in need thereof, comprising: a) identifying a subject in need of increased insulin sensitivity; and b) administering to the subject a composition comprising an effective amount of a peptide, wherein the peptide comprises 10 amino acids or less in length, wherein about 50% of the amino acids or greater are amino acids with a carboxylate side chain or amino acids with a side chain that can be converted to a carboxylate side chain, and wherein the peptide has chromium binding activity.

10. The method of claim 9, wherein the peptide comprises an amino acid sequence according to the formula XXXXGXX (SEQ ID NO: 4), wherein X is an amino acid with a carboxylate side chain or an amino acid with a side chain that can be converted to a carboxylate side chain.

11. The method of claim 10, wherein the peptide is a peptide comprising SEQ ID NO: 3.

12. The method of claim 11, wherein the peptide is a peptide consisting of SEQ ID NO: 3.

13. The method of claim 9, further comprising administering chromium to the subject.

14. The method of claim 9, further comprising administering an effective amount of insulin to the subject.

15. The method of claim 14, wherein the effective amount of insulin administered to the subject is lower than the diabetic dosage of insulin administered to the subject in the absence of the peptide.

16. The method of claim 9, further comprising administering an effective amount of a non-insulin therapeutic agent to the subject.

17. The method of claim 16, wherein the therapeutic agent is selected from the group consisting of biguanines, sulfonylureas, meglitinides, thiazolidinediones, dipeptidyl peptidase-4 inhibitors and glucagon-like peptide-1 receptor agonists.

18. The method of claim 9, wherein the subject has Type 2 diabetes.

19. The method of claim 9, wherein the peptide does not comprise pyroglutamate.

20. A synergistic pharmaceutical combination comprising:
a) a first pharmaceutical composition comprising an anti-diabetic agent; and
b) a second pharmaceutical composition comprising a peptide, wherein the peptide comprises 10 amino acids or less in length, wherein about 50% of the amino acids or greater are amino acids with a carboxylate side chain or amino acids with a side chain that can be converted to a carboxylate side chain, and wherein the peptide has chromium binding activity.

21. The pharmaceutical combination of claim 20, wherein the effective dosage of the anti-diabetic when used in combination with the peptide is less than the effective dosage of the anti-diabetic when used alone.

22. The pharmaceutical combination of claim 20, wherein the peptide and the anti-diabetic are in separate formulations.

23. The pharmaceutical combination of claim 22, wherein the formulations are unit dosage formulations.

24. The pharmaceutical combination of claim 22, wherein the peptide formulation further comprises chromium.

25. The method of claim 20, wherein the anti-diabetic agent is insulin.

26. The method of claim 20, wherein the peptide is a peptide comprising or consisting of SEQ ID NO: 3.

27. The method of claim 20, wherein the peptide does not comprise pyroglutamate.


SEQUENCE LISTING (42 sequences)

SEQ ID NO: 1 - protein, 8 aa, Artificial Sequence
  Synthetic Construct
XEEEEGDD

SEQ ID NO: 2 - protein, 8 aa, Artificial Sequence
  Synthetic Construct
XEEEGEDD

SEQ ID NO: 3 - protein, 7 aa, Artificial Sequence
  Synthetic Construct
EEEEGDD

SEQ ID NO: 4 - protein, 7 aa, Artificial Sequence
  Synthetic Construct
XXXXGXX

SEQ ID NO: 5 - protein, 7 aa, Artificial Sequence
  Synthetic Construct
EEEEGNN

SEQ ID NO: 6 - protein, 7 aa, Artificial Sequence
  Synthetic Construct
EEEEGDN

SEQ ID NO: 7 - protein, 7 aa, Artificial Sequence
  Synthetic Construct
EEEEGND

SEQ ID NO: 8 - protein, 7 aa, Artificial Sequence
  Synthetic Construct
QEEEGDD

SEQ ID NO: 9 - protein, 7 aa, Artificial Sequence
  Synthetic Construct
EQEEGDD

SEQ ID NO: 10 - protein, 7 aa, Artificial Sequence
  Synthetic Construct
EEQEGDD

SEQ ID NO: 11 - protein, 7 aa, Artificial Sequence
  Synthetic Construct
EEEQGDD

SEQ ID NO: 12 - protein, 7 aa, Artificial Sequence
  Synthetic Construct
QQEEGDD

SEQ ID NO: 13 - protein, 7 aa, Artificial Sequence
  Synthetic Construct
QEEQGDD

SEQ ID NO: 14 - protein, 7 aa, Artificial Sequence
  Synthetic Construct
EQQEGDD

SEQ ID NO: 15 - protein, 7 aa, Artificial Sequence
  Synthetic Construct
EQEQGDD

SEQ ID NO: 16 - protein, 7 aa, Artificial Sequence
  Synthetic Construct
EEQQGDD

SEQ ID NO: 17 - protein, 7 aa, Artificial Sequence
  Synthetic Construct
QQQEGDD

SEQ ID NO: 18 - protein, 7 aa, Artificial Sequence
  Synthetic Construct
EQQQGDD

SEQ ID NO: 19 - protein, 8 aa, Artificial Sequence
  Synthetic Construct
XEEEEGNN

SEQ ID NO: 20 - protein, 8 aa, Artificial Sequence
  Synthetic Construct
XEEEEGDN

SEQ ID NO: 21 - protein, 8 aa, Artificial Sequence
  Synthetic Construct
XEEEEGND

SEQ ID NO: 22 - protein, 8 aa, Artificial Sequence
  Synthetic Construct
XQEEEGDD

SEQ ID NO: 23 - protein, 8 aa, Artificial Sequence
  Synthetic Construct
XEQEEGDD

SEQ ID NO: 24 - protein, 8 aa, Artificial Sequence
  Synthetic Construct
XEEQEGDD

SEQ ID NO: 25 - protein, 8 aa, Artificial Sequence
  Synthetic Construct
XEEEQGDD

SEQ ID NO: 26 - protein, 8 aa, Artificial Sequence
  Synthetic Construct
PQQEEGDD

SEQ ID NO: 27 - protein, 8 aa, Artificial Sequence
  Synthetic Construct
XQEEQGDD

SEQ ID NO: 28 - protein, 8 aa, Artificial Sequence
  Synthetic Construct
XEQQEGDD

SEQ ID NO: 29 - protein, 8 aa, Artificial Sequence
  Synthetic Construct
XEQEQGDD

SEQ ID NO: 30 - protein, 8 aa, Artificial Sequence
  Synthetic Construct
XEEQQGDD

SEQ ID NO: 31 - protein, 8 aa, Artificial Sequence
  Synthetic Construct
XQQQEGDD

SEQ ID NO: 32 - protein, 8 aa, Artificial Sequence
  Synthetic Construct
XEQQQGDD

SEQ ID NO: 33 - protein, 7 aa, Artificial Sequence
  Synthetic Construct
DEEEGDE

SEQ ID NO: 34 - protein, 7 aa, Artificial Sequence
  Synthetic Construct
EDEEGDE

SEQ ID NO: 35 - protein, 7 aa, Artificial Sequence
  Synthetic Construct
EEDEGDE

SEQ ID NO: 36 - protein, 7 aa, Artificial Sequence
  Synthetic Construct
EEEDGDE

SEQ ID NO: 37 - protein, 7 aa, Artificial Sequence
  Synthetic Construct
DEEEGED

SEQ ID NO: 38 - protein, 7 aa, Artificial Sequence
  Synthetic Construct
EDEEGED

SEQ ID NO: 39 - protein, 7 aa, Artificial Sequence
  Synthetic Construct
EEDEGED

SEQ ID NO: 40 - protein, 7 aa, Artificial Sequence
  Synthetic Construct
EEEDGED

SEQ ID NO: 41 - protein, 10 aa, Artificial Sequence
  Synthetic Construct
EDGEECDCGE

SEQ ID NO: 42 - protein, 10 aa, Artificial Sequence
  Synthetic Construct
DGEECDCGEE